(19) United States
(12) Patent Application Publication
(10) Pub. No.: US 2024/0079090 A1
(43) Pub. Date: Mar. 7, 2024

(54) VISUALIZATION OF SYNTHETICALLY-MODIFIED MOLECULAR NUCLEOTIDE SEQUENCES

(71) Applicant: Benchling, Inc., San Francisco, CA (US)

(72) Inventors: Pathikrit Bhattacharyya, Oakland, CA (US); Andrew Yi Guo, Fremont, CA (US); Tommy Li, San Mateo, CA (US); Naomi Anna Jacobs, San Francisco, CA (US); Henry Marfleet Willson, New York, NY (US); Alan Garrett Pierce, San Francisco, CA (US)

(21) Appl. No.: 17/939,667

(22) Filed: Sep. 7, 2022

(51) Int. Cl. G16B 30/00; G16B 40/20; G16B 45/00

(52) U.S. Cl. CPC G16B 30/00; G16B 40/20; G16B 45/00

(57) ABSTRACT

Methods, systems, and apparatus, including computer programs encoded on a computer storage medium, for updating a molecular database. In one aspect, a method includes: receiving data representing a sequence of molecular nucleotides, where the data specifies a base of each molecular nucleotide and synthetic modifications to one or more components of one or more molecular nucleotides in the sequence; generating a user interface presentation that presents: i) for each base in the sequence, a base character that represents the base of the molecular nucleotide, and ii) for each of the one or more molecular nucleotides having one or more components that are synthetically modified, a group of symbols adjacent to the base character that represents the base of the molecular nucleotide, where each symbol in the group of symbols represents a respective synthetic modification; and providing the user interface presentation to a user device for display to a user.

### BACKGROUND

This specification relates to visualization of molecular nucleotide sequences.

A molecule can refer to a group of bonded atoms. Examples of molecules include deoxyribonucleic acid (DNA) molecules, ribonucleic acid (RNA) molecules (e.g., messenger RNA) xeno nucleic acid (XNA) molecules, protein molecules, peptide molecules, antibody molecules, drug molecules, antibody-drug conjugate molecules, carbohydrate molecules, and lipid molecules. Other examples of molecules include oligonucleotides that are short DNA or RNA molecules having a wide range of applications in genetic testing, scientific research, and forensics. Examples of oligonucleotides include microRNA (miRNA), small interfering RNA (siRNA), small activating RNA (saRNA), antisense oligonucleotides (ASOs), and aptamers.

Certain molecules, e.g., DNA, RNA, and oligonucleotides, can be referred to as being defined by a sequence of molecular nucleotides. A molecular nucleotide can refer to a monomeric unit of a molecule. For example, the RNA molecule can consist of a sequence of molecular nucleotides, where each molecular nucleotide includes three components: a sugar (e.g., ribose), a phosphate, and a nitrogenous base (e.g., guanine, uracil, adenine, or cytosine). The components of the molecular nucleotides of a molecule (e.g., a naturally-occurring molecule, such as RNA) can be synthetically modified to evoke a desired behavior of the molecule, e.g., to improve its stability and functionality. These synthetically modified molecules can then be used for various applications, including delivery of treatments targeting diseases that are difficult to address with other therapeutic approaches.

Data representing molecular nucleotide sequences can be electronically stored in a database and used to generate molecular nucleotide sequence visualizations. These visualizations can support scientists and researchers in developing new therapeutic treatments.

### SUMMARY

This specification describes a bioinformatics platform implemented as computer programs on one or more computers in one or more locations that can generate a user interface presentation for visualizing sequences of molecular nucleotides.

The bioinformatics platform can be a distributed cloud-based computing system where multiple users and laboratories can upload and store sequence data defining sequences of molecular nucleotides. The sequence data can specify one or more components of each of the molecular nucleotides in the sequence. In some cases, the sequence data can further specify synthetic modifications to one or more components of the molecular nucleotides in the sequence. Throughout this specification, a “synthetic modification” of a component of a molecular nucleotide can generally refer to any appropriate chemical modification to the component of the molecular nucleotide. In this context, the platform can consider a component to be synthetically modified when it differs from an original state. What the system considers to be the original state can vary depending on the application and can be user-customizable. Synthetic modifications thus include human-induced modifications to the chemical structure of molecular nucleotides relative to an original state of the molecular nucleotides. Synthetic modifications thus encompass exogenous, non-naturally occurring modifications, as well as modifications to sequences that result in structures that might be naturally occurring elsewhere.

The bioinformatics platform can process sequence data and generate a user interface presentation that efficiently represents the sequence of molecular nucleotides and associated synthetic modifications. In some implementations, the platform can efficiently present sequences of variable lengths, e.g., sequences that include 10 molecular nucleotides, 100 molecular nucleotides, 1000 molecular nucleotides, 5000 molecular nucleotides, or any other appropriate number of molecular nucleotides. In each of these cases, the bioinformatics platform is able to adjust the user interface presentation so as to efficiently represent the synthetic modifications to one or more components of the molecular nucleotides in the sequence.

According to a first aspect, there is provided a computer-implemented method that includes: receiving data representing a sequence of molecular nucleotides, where the data specifies a base of each molecular nucleotide in the sequence of molecular nucleotides and synthetic modifications to one or more components of one or more molecular nucleotides in the sequence of molecular nucleotides, generating a user interface presentation that presents: i) for each base in the sequence of molecular nucleotides, a base character that represents the base of the molecular nucleotide, and ii) for each of the one or more molecular nucleotides having one or more components that are synthetically modified, a group of symbols adjacent to the base character that represents the base of the molecular nucleotide, where each symbol in the group of symbols represents a respective synthetic modification, and providing the user interface presentation to a user device for display to a user.

In some implementations, the group of symbols is presented as a group of vertically stacked symbols.

In some implementations, the group of symbols is presented above the base character that represents the base of the molecular nucleotide.

In some implementations, the group of vertically stacked symbols includes visually distinguishable symbols to represent different synthetic modifications.

In some implementations, each symbol included in the group of vertically stacked symbols has a respective shape to represent the respective synthetic modification that is different from the shapes of the other symbols in the group of vertically stacked symbols.

In some implementations, the one or more components that are synthetically modified comprise a sugar and a phosphate, and where the synthetic modification to the sugar is represented using a symbol having a first shape and the synthetic modification to the phosphate is represented using a symbol having a second different shape.

In some implementations, the one or more components that are synthetically modified further comprise the base of the molecular nucleotide, and where the synthetic modification to the base is represented using a symbol having a fill pattern that is different from a fill pattern of the symbol representing the synthetic modification to the sugar and a fill pattern of the symbol representing the synthetic modification to the phosphate.

In some implementations, the method further includes: receiving a request to zoom in within the user interface presentation, and in response, transitioning the user interface presentation to a different display format.

In some implementations, the one or more components that are synthetically modified comprise a sugar, and where the synthetic modification to the sugar is represented in the different display format using an outline of the base character that represents the base of the molecular nucleotide.

In some implementations, the one or more components that are synthetically modified further comprise the base of the molecular nucleotide, and where the synthetic modification to the base is represented in the different display format using a shaded background of the base character that represents the base of the molecular nucleotide.

In some implementations, the one or more components that are synthetically modified further comprise a phosphate, and where the synthetic modification to the phosphate is represented in the different display format using a single symbol adjacent to the base character that represents the base of the molecular nucleotide.

In some implementations, the method further includes: obtaining user-specified visual customizations for the synthetic modifications to the one or more components of the one or more molecular nucleotides in the sequence of molecular nucleotides, and updating the user interface presentation or the different display format of the user interface presentation according to the user-specified visual customizations.

In some implementations, the user-specified visual customizations specify different display colors for different types of synthetic modifications to the one or more components of the one or more molecular nucleotides in the sequence of molecular nucleotides.

In some implementations, the data representing the sequence of molecular nucleotides is in a first format, and where the method further includes: receiving a user-specified mapping between the first format and a second format; and automatically converting the data into the second format according to the user-specified mapping.

In some implementations, the method further includes: receiving a request to compare the sequence of molecular nucleotides to a second sequence of molecular nucleotides, determining that a first nucleotide in the sequence of molecular nucleotides has a synthetic modification that is not present in a second nucleotide at a corresponding position in the second sequence of molecular nucleotides, and representing in the user interface presentation the synthetic modification that is not present in the second nucleotide using a symbol that is visually distinguished from other symbols in the user interface presentation.

In some implementations, the user interface presentation presents user-customizable grouping information for subsequences of the sequence of molecular nucleotides.

In some implementations, the user interface presentation includes user interface elements for editing labels for the subsequences and a beginning and an end of the subsequences of the sequence of molecular nucleotides.

In some implementations, the method further includes: presenting a user interface control for selecting one or more additional synthetic modifications, receiving a selection to present the one or more additional, or alternative, synthetic modifications, and in response to receiving the selection, modifying the group of symbols adjacent to the base character that represents the base of the molecular nucleotide to represent the one or more additional, or alternative, synthetic modifications.

According to a second aspect, there is provided a system including: one or more computers; and one or more storage devices communicatively coupled to the one or more computers, where the one or more storage devices store instructions that, when executed by the one or more computers, cause the one or more computers to perform the operations of the respective method of any preceding aspect.

According to a third aspect, there are provided one or more non-transitory computer storage media storing instructions that when executed by one or more computers cause the one or more computers to perform the operations of the respective method of any preceding aspect.

Particular embodiments of the subject matter described in this specification can be implemented so as to realize one or more of the following advantages.

The bioinformatics platform described in this specification can generate user interface presentations that efficiently present various synthetic modifications to individual components of molecular nucleotides included in molecular nucleotide sequences. For example, a group of vertically stacked symbols above a base character can represent various synthetic modifications in an easily-interpretable and distinguishable manner, such that a user of the bioinformatics platform can quickly and efficiently understand the sequence information being presented. Moreover, the bioinformatics platform described in this specification can be modified using a variety of different user-specified visual customizations to flexibly adjust the user interface presentation as desired. For example, the bioinformatics platform allows users to specify visual customizations to synthetic modifications and grouping information for subsequences, thereby facilitating a much more accessible and efficient interpretability of the sequence data.

Furthermore, the bioinformatics platform described in this specification can adaptively adjust the user interface presentation to suit a sequence of a particular length. For example, a user of the bioinformatics platform can switch the user interface presentation (e.g., using a zoom function) from a first format of the user interface presentation that is suitable for presentation of sequences including, e.g., less than 100 molecular nucleotides in length, to a second format of the interface presentation that is suitable for presentation of longer sequences including, e.g., 4000 molecular nucleotides. In this manner, a user of the bioinformatics platform can switch the user interface presentation between different display formats while preserving the same amount of information regarding synthetic modifications. Therefore, the bioinformatics platform can leverage the rich representational capacity of synthetic modifications while enabling efficient presentation of sequences of variable lengths.

The bioinformatics platform described in this specification can facilitate bulk editing of molecular nucleotide sequences in the user interface presentation. For example, a user can select multiple molecular nucleotides that satisfy a particular criterion (e.g., that have the same base, the same synthetic modification, or any other appropriate criterion), and simultaneously modify the representations of these molecular nucleotides in the user interface presentation. In some cases, a user can use a “find and replace” function of the bioinformatics platform to find, select, and edit particular molecular nucleotides in the sequence. In some cases, a user can select molecular nucleotides at particular positions in the sequence. In this manner, the bioinformatics platform enables efficient analysis, search, and editing of molecular nucleotide sequences of any appropriate length.

Moreover, the bioinformatics platform described in this specification can automatically convert sequence data from any appropriate format into a format that is used for storage in a sequence database of the bioinformatics platform. Therefore, the bioinformatics platform can maintain consistency of sequence data stored in the sequence database. Any user/laboratory can use the bioinformatics platform by specifying an appropriate mapping between the format of the sequence data adopted by the user/laboratory and the format of the sequence data stored in the sequence database. In this manner, the bioinformatics platform can be universally accessible to, and usable by, a variety of different entities and data sources, without the need for adaptation and redesign.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example bioinformatics platform that can generate a user interface presentation of a sequence of molecular nucleotides.

FIG. 2 illustrates an example sequence of molecular nucleotides.

FIG. 3 illustrates an example user interface presentation generated by a bioinformatics platform.

FIG. 4 illustrates a different display format of the example user interface presentation generated by the bioinformatics platform.

FIG. 5 illustrates example user interface controls for selecting additional, or alternative, synthetic modifications to present in the user interface presentation.

FIG. 6 is a flow diagram of an example process for generating a user interface presentation of a sequence of molecular nucleotides.

Like reference numbers and designations in the various drawings indicate like elements.

### DETAILED DESCRIPTION

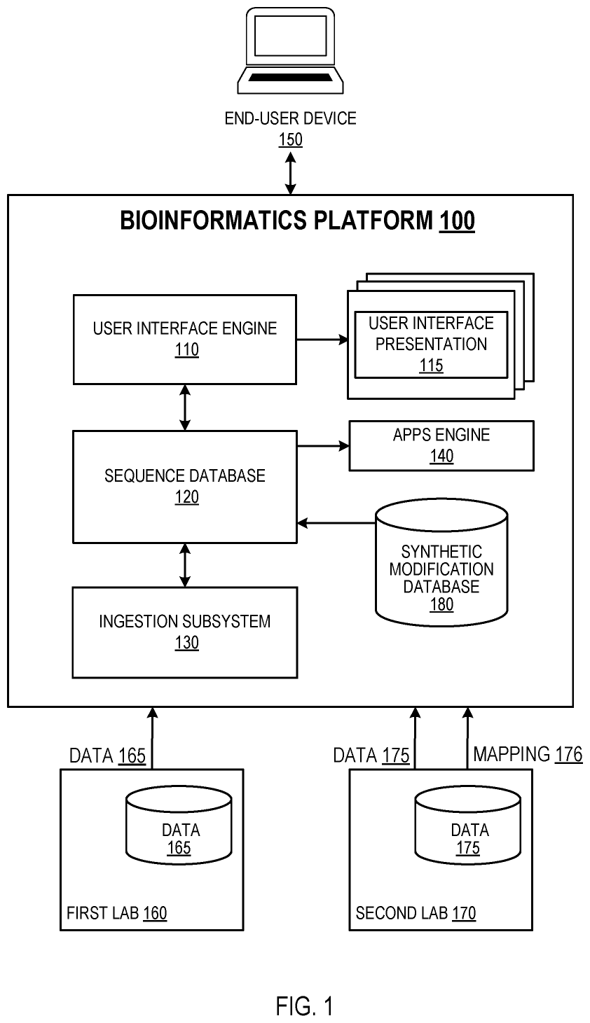

FIG. 1 is a block diagram of an example bioinformatics platform 100 that can generate a user interface presentation 115 of a sequence of molecular nucleotides. The bioinformatics platform 100 is an example of a system implemented as computer programs on one or more computers in one or more locations in which the systems, components, and techniques described below are implemented.

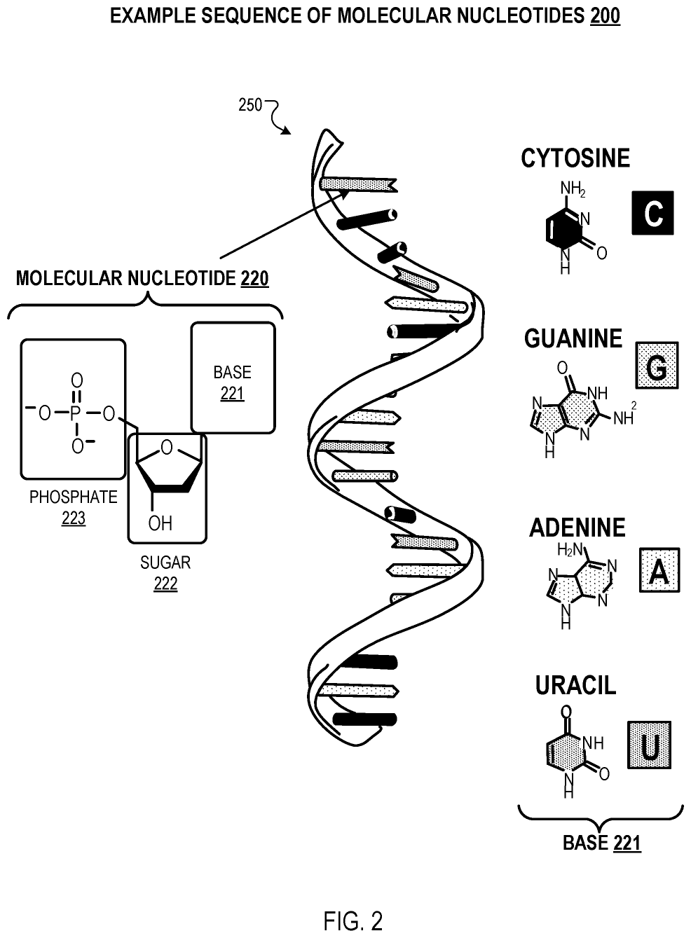

The bioinformatics platform 100 can receive data 165 representing a sequence of molecular nucleotides and generate the user interface presentation 115 of the sequence. As described in more detail below with reference to FIG. 2, the sequence of molecular nucleotides can represent a molecule, e.g., deoxyribonucleic acid (DNA) molecule, a ribonucleic acid (RNA) molecule, an oligonucleotide, or any other appropriate molecule. For example, the naturally-occurring RNA molecule can be represented as a sequence of molecular nucleotides that each include three components: a sugar (e.g., ribose), a phosphate, and a nitrogenous base (e.g., guanine, uracil, adenine, or cytosine). Generally, the sequence data 165 can specify the sequence of molecular nucleotides in any appropriate format, e.g., using Hierarchical Editing Language for Macromolecules (HELM).

In some cases, the data 165 (also referred to as sequence data) can specify a base of each molecular nucleotide in the sequence and synthetic modifications to one or more components of one or more molecular nucleotides in the sequence. Throughout this specification, a “component” of a molecular nucleotide can generally refer to any subunit of the molecular nucleotide, e.g., a phosphate in a molecular nucleotide of the RNA molecule. Further, a “synthetic modification” of a component of the molecular nucleotide can generally refer to any appropriate number and type of chemical modifications to the component of the molecular nucleotide. Moreover, the “synthetic modification” of a component of the molecular nucleotide can include any type of exogenous and endogenous modification to the component of the molecular nucleotide. In the case of the RNA molecule, a “synthetic modification” can include a modification to one or more of: the sugar, the phosphate, and the nitrogenous base.

Throughout this specification, a molecule that is represented by a sequence of molecular nucleotides, where one or more components of at least one molecular nucleotide in the sequence are synthetically modified, can be referred to as a “synthetic” molecule. For example, the synthetic molecule can be a synthetic messenger RNA (mRNA) molecule having synthetically-modified bases that are specifically designed to improve the effectiveness of mRNA vaccine against infectious disease.

The bioinformatics platform 100 can be a distributed cloud-based computing system that includes: (i) a user interface engine 110, (ii) a sequence database 120, (iii) an ingestion system 130, (iv) an apps engine 140, and (v) a synthetic modification database 180, each of which is described in more detail next.

The ingestion subsystem 130 can be configured to receive the sequence data 165 and sore it in the sequence database 120. The ingestion subsystem 130 can receive the sequence data 165 from, e.g., a first laboratory 160, a second laboratory 170, a user of an end-user device 150, or in any other appropriate manner. As a particular example, the user of the end-user device 150 can provide the sequence data 165 by way of an input into a user interface (e.g., a graphical user interface, GUI), or an application programming interface (API), made available by the bioinformatics platform 100 or the end-user device 150. As another particular example, the ingestion subsystem 130 can receive the sequence data 165 through a network, which can be a local area network (LAN), a wide area network (WAN), the Internet, or a combination thereof, from one or more laboratories 160, 170.

Generally, the ingestion subsystem 130 can be configured to receive the sequence data 165 in any appropriate format, while the sequence database 120 can be configured to store the sequence data 165 in a particular database format e.g., Hierarchical Editing Language for Macromolecules (HELM), or any other appropriate format.

In some implementations, the ingestion subsystem 130 can be configured to receive the sequence data 175 from a second laboratory 170 that uses a laboratory format, e.g., a format that uses a different notation to specify molecular nucleotide sequences than the database format used by the sequence database 120. In such cases, the ingestion subsystem 130 can additionally receive mapping data 176, e.g., a user-specified mapping between the laboratory format and the database format. The ingestion subsystem 130 can process the sequence data 175 in the laboratory format and the mapping 176 and automatically convert the sequence data 175 into the database format according to the mapping. After converting the sequence data 175 into the database format, the ingestion subsystem 130 can store the sequence data 175 in the database format in the sequence database 120. In some cases, in addition to user-specified mapping between formats, mapping data 176 can further include user-specified “original” states of molecular nucleotides, e.g., states relative to which molecular nucleotides are synthetically modified. In other words, mapping data 176 can specify, for each molecular nucleotide, a state of the molecular nucleotide before human-induced modification, exogenous, non-naturally occurring modification, naturally-occurring modification, or any other appropriate type of modification to its chemical structure.

By automatically converting the data 175 based on the mapping 176 into the database format, the ingestion subsystem 130 can maintain the consistency of sequence data stored in the sequence database 120. Moreover, the ingestion subsystem 130 can enable multiple users/laboratories to upload sequence data to the bioinformatics platform 100 irrespective of the format of the sequence data adopted by the users/laboratories. Furthermore, any type of laboratory is able to upload data to the bioinformatics platform 100 by specifying an appropriate mapping 176 between the respective laboratory format and the database format.

The sequence database 120 can include data that specifies any appropriate number of molecular nucleotide sequences. For example, the sequence database 120 can include tens, hundreds, thousands, tens of thousands, or hundreds of thousands of molecular nucleotide sequences. Moreover, the sequence database 120 can store data specifying sequences of molecular nucleotides representing multiple types of molecules. For example, the sequence database 120 can simultaneously store one or more molecular nucleotide sequences representing DNA molecules and one or more molecular nucleotide sequences representing RNA molecules. In addition to the sequence database 120 that stores sequence data 165, 175, the bioinformatics platform can further include the synthetic modification database 180 that can store data specifying different types of possible synthetic modifications to various molecular nucleotides. The bioinformatics platform 100 can obtain the synthetic modification data that is stored in the synthetic modification database 180 from scientific literature, or in any other appropriate manner. In some cases, the synthetic modifications database 180 can store synthetic modification data in the same format as the sequence database 120, e.g., Hierarchical Editing Language for Macromolecules (HELM), or any other appropriate format.

The bioinformatics platform 100 can further include the apps engine 140 that is configured to carry out one or more tasks associated with the sequence data 165. In some implementations, the apps engine 140 can include one or more software application programs executable within the bioinformatics platform 100 that can analyze the sequence data 165. In some implementations, the apps engine 140 can further include a machine learning logic that can be any appropriate machine learning algorithm, e.g., linear regression, logistic regression, Bayes classifiers, random classifiers, decision trees, and any other appropriate machine learning algorithm, or a combination thereof. The apps engine 140 can further include a training logic that can train the machine learning logic to analyze the sequence data 165. In some cases, a user of the end-user device 150 can use the apps engine 140 to analyze the sequence data 165 stored in the sequence database 120. The bioinformatics platform 100 can therefore facilitate an efficient analysis of legacy data in any appropriate format that may otherwise be too computationally-expensive, or difficult, to perform. Furthermore, the bioinformatics platform 100 can make the analysis results accessible from any appropriate device (e.g., the end-user device 150).

The bioinformatics platform 100 further includes the user interface engine 110 that is configured to generate the user interface presentation 115 of the molecular nucleotide sequence specified by the sequence data 165. As described in more detail below with reference to FIG. 2, the molecular nucleotide sequence can represent, e.g., an RNA molecule, where each molecular nucleotide in the sequence includes three components: a sugar, a phosphate, and a base. The sequence data 165 can specify a base of each molecular nucleotide in the sequence of molecular nucleotides. For example, in the case of the RNA molecule, the base can include one of: guanine (G), uracil (U), adenine (A), or cytosine (C). As another example, in the case of the DNA molecule, the base can include one of: guanine (G), thymine (T), adenine (A), or cytosine (C). The sequence data 165 can further specify one or more synthetic modifications to one or more of these components in the sequence of molecular nucleotides. As a particular example, the sequence of molecular nucleotides can include a first molecular nucleotide with a synthetically-modified sugar, a second molecular nucleotide with a synthetically modified base (e.g., synthetically-modified adenine (A)), and a third molecular nucleotide that does not include any synthetically-modified components.

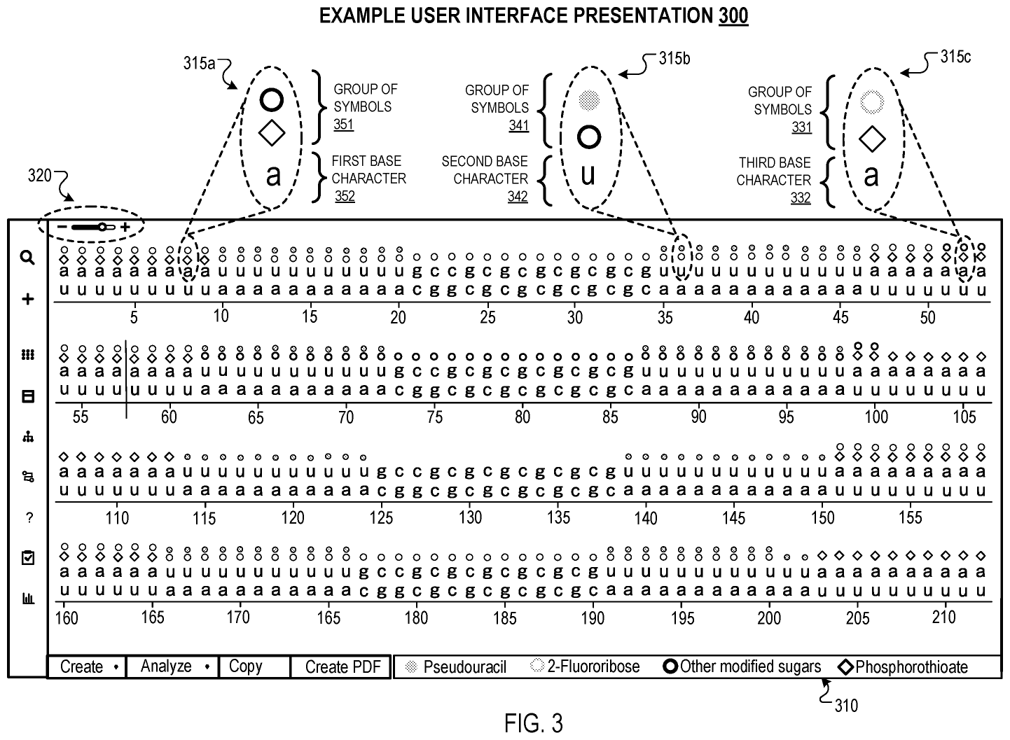

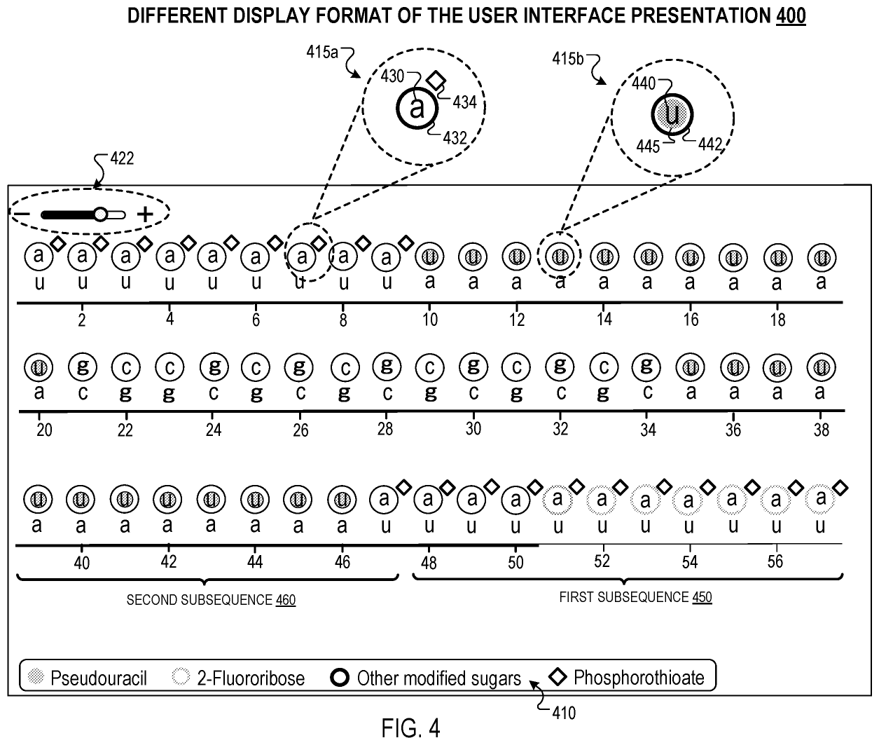

The user interface engine 110 can generate the user interface presentation 110 that presents, for each base in the sequence of molecular nucleotides, a base character that represents the base of the molecular nucleotide (e.g., “G” for guanine, “U” for uracil, “A” for adenine, and “C” for cytosine). The user interface presentation 115 can also present, for each of the one or more molecular nucleotides having one or more components that are synthetically modified, a group of symbols adjacent to the base character that represents the base of the molecular nucleotide. For example, for the second molecular nucleotide in the sequence having the synthetically-modified adenine (A), the user interface presentation 115 can present the base character “A” and a group of symbols adjacent to the base character indicating the chemical modification of the adenine. Each of the symbols in the group can represent a respective synthetic modification. Moreover, the group of symbols can generally include any appropriate number of symbols, e.g., one symbol, two symbols, three symbols, or any other appropriate number of symbols. An example user interface presentation is described in more detail below with reference to FIG. 3 and FIG. 4. Although the above example is described with respect to a molecular nucleotide sequence that represents a synthetic RNA molecule, the user interface engine 110 can generate the user interface presentation 115 for any appropriate sequence of molecular nucleotides representing any appropriate molecule.

The bioinformatics platform 100 can provide the user interface presentation 115 for display to a user of the end-user device 150. Generally, the end-user device 150 can be an electronic device that is capable of requesting and receiving content over the network described above, e.g., the Internet. The end-user device 150 can include any client computing device such as a laptop/notebook computer, wireless data port, smart phone, personal data assistant (PDA), tablet computing device, one or more processors within these devices, or any other suitable processing device that can send and receive data over the network. For example, the end-user device 150 can include, e.g., a computer that includes an input device, such as a keypad, touch screen, or other device that can accept user information, and an output device that conveys information, including digital data, visual information, and/or the user interface presentation 115. The end-user 150 can include one or more client applications. A client application is any type of application that allows the end-user device 150 to request and view content on a respective client device. In some implementations, a client application can use parameters, metadata, and other information received, e.g., at launch, to access a particular set of data from the bioinformatics platform 100.

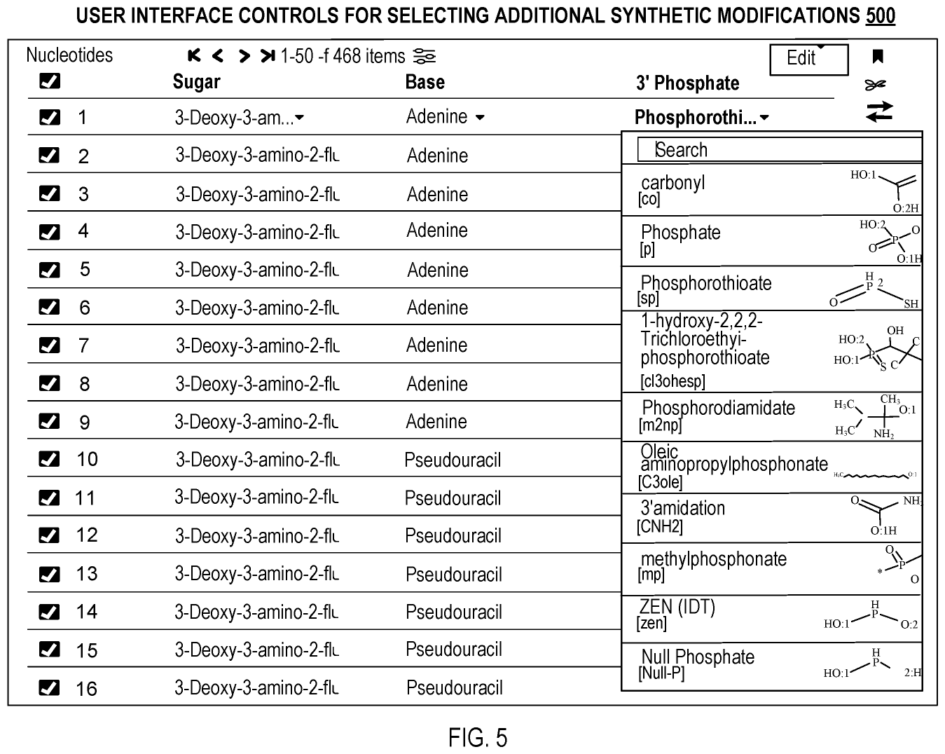

As described in more detail below with reference to FIG. 3, FIG. 4, and FIG. 5, a user of the end-user device 150 can view the user interface presentation 115 and modify the user interface presentation 115 using one or more controls presented in the user interface presentation 115. For example, the user can interact with the controls to select one or more additional (or alternative) synthetic modifications from the synthetic modification database 180. After receiving the user selection, the user interface engine 110 can modify the user interface presentation 115 to present the additional (or alternative) synthetic modifications selected by the user. In some cases, a user of the end-user device 150 can specify visual customizations for the synthetic modifications, and the user interface engine 110 can accordingly modify the user interface presentation 115.

In some implementations, a user of the end-user device 150 can compare different sequences of molecular nucleotides. For example, a user can use the end-user device 150 to provide a request to the bioinformatics platform 100 to compare the sequence of molecular nucleotides specified by the sequence data 165 and/or presented in the user interface presentation 115 to a second sequence of molecular nucleotides (e.g., included in the sequence database 120). The bioinformatics platform 100 can compare the sequences and determine, e.g., that a first nucleotide in the sequence of molecular nucleotides has a synthetic modification that is not present in a second nucleotide at a corresponding position in the second sequence of molecular nucleotides.

For example, the bioinformatics platform 100 can determine that the molecular nucleotide at a fifth position in the sequence represented by the sequence data 165 includes a synthetic modification to the sugar of the molecular nucleotide, whereas the molecular nucleotide at the fifth position in the second sequence does not include the synthetic modification to the sugar of the molecular nucleotide. Based on this determination, the bioinformatics platform 100 can use the user interface engine 110 to present in the user interface presentation 115 the synthetic modification that is not present in the second nucleotide using a symbol that is visually distinguished from other symbols in the user interface presentation. For example, the user interface engine 115 can modify the shape/color, or otherwise distinguish, the symbol representing the synthetic modification to the sugar of the molecular nucleotide at the fifth position in the user interface presentation 115 from the other symbols in the user interface presentation in order to, e.g., alert the user that this synthetic modification is not present in the second sequence.

An example sequence of molecular nucleotides is described in more detail next.

FIG. 2 illustrates an example sequence of molecular nucleotides 200. In the example illustrated in FIG. 2, the sequence of molecular nucleotides represents an RNA molecule 250. Each molecular nucleotide 220 in the sequence can include three components: a base 221, a phosphate 223, and a sugar 222. The base 221 of each molecular nucleotide can include one of: cytosine (C), guanine (G), adenine (A) and uracil (U). As described above with reference to FIG. 1, a bioinformatics platform can process sequence data specifying the sequence of molecular nucleotides 200 representing the molecule 250 and generate a user interface presentation that presents, for each base 221 in the sequence 200, a base character that represents the base 221 of the molecular nucleotide (e.g., “C” for cytosine). As a particular example, if the sequence of molecular nucleotides 200 includes a first molecular nucleotide having a cytosine base, a second molecular nucleotide having an adenine base, and a third molecular nucleotide having a uracil base, the user interface presentation can present “C,” followed by “A,” followed by “U.”

The user interface presentation can further present, for each of the one or more molecular nucleotides in the sequence 200 having one or more components that are synthetically modified, a group of symbols adjacent to the base character that represents the base of the molecular nucleotide. Each symbol in the group of symbols represents a respective synthetic modification. As a particular example, if the first molecular nucleotide in the sequence 200 having the cytosine base includes a synthetically-modified phosphate, and a synthetically-modified sugar, the user interface presentation can present the group of symbols next to the base character “C,” where each symbol represents the respective synthetic modification.

Example user interface presentations are described in more detail below with reference to FIG. 3, FIG. 4, and FIG. 5.

FIG. 3 illustrates an example user interface presentation 300 (e.g., the user interface presentation 115 in FIG. 1) generated by the bioinformatics platform of FIG. 1. In the example of FIG. 3, the user interface presentation 300 presents a sequence of molecular nucleotides that includes 212 molecular nucleotides, each molecular nucleotide having a corresponding position in the sequence indicated by a number, e.g., 1, 2, 3, etc. In general, the user interface presentation 300 can present a sequence of molecular nucleotides having any appropriate length, e.g., 10 molecular nucleotides, 100 molecular nucleotides, 1000 molecular nucleotides, 5000 molecular nucleotides, or any other appropriate number of molecular nucleotides. FIG. 3 illustrates exploded views 315a, 315b, and 315c for clarity.

As illustrated in FIG. 3, the user interface presentation 300 presents, for each base in the sequence of molecular nucleotides, a base character that represents the base of the molecular nucleotide. For example, a first base character 352 (“a”) represents a molecular nucleotide (e.g., positioned at the 8^{th} position in the molecular nucleotide sequence) having adenine base. A second base character 342 (“u”) represents a molecular nucleotide (e.g., positioned at the 36^{th} position in the molecular nucleotide sequence) having uracil base. A third base character 332 (“a”) represents a molecular nucleotide (e.g., positioned at the 52^{nd} position in the molecular nucleotide sequence) having adenine base.

The user interface presentation 300 also presents, for each of one or more molecular nucleotides having one or more components that are synthetically modified, a group of symbols adjacent to the base character that represents the base of the molecular nucleotide. The group of symbols can generally include any appropriate number of symbols, e.g., a single symbol, two symbols, three symbols, four symbols, five symbols, or any other appropriate number of symbols. As illustrated in FIG. 3, the group of symbols can be presented as a group of vertically stacked symbols. The user interface presentation 300 can leverage the rich representational capacity of the group of vertically stacked symbols to present a wealth of information in a compact and intuitive way. Moreover, the group of symbols can be presented above the base character that represents the base of the molecular nucleotide. For example, the group of symbols 351 representing synthetic modifications to one or more components of the molecular nucleotide the base of which is represented using the first base character 352 is presented above the base character 352 (e.g., “a”) in the user interface presentation 300.

The group of symbols can include visually distinguishable symbols to represent different synthetic modifications. For example, each symbol in the group of vertically stacked symbols can have a respective shape to represent the respective synthetic modification that is different from the shapes of the other symbols in the group. Generally, the symbols in the group of symbols can be visually distinguished in any appropriate manner, e.g., using different shapes, colors, shading, font style, or any other appropriate manner.

As a particular example, the group of symbols 351 above the first base character 352 (e.g., “a”) includes a first symbol, e.g., an empty circle with a black outline, and a second symbol, e.g., a rhombus. The circle with the black outline represents a synthetic modification to the sugar of the molecular nucleotide, while the rhombus represents a phosphorothioate synthetic modification to the phosphate of the molecular nucleotide. Similarly, the group of symbols 331 above the third base character 332 (e.g., “a”) includes a first symbol, e.g., an empty circle with a gray outline, and a second symbol, e.g., a rhombus. In this case, the rhombus above the third base character 332 represents the same synthetic modification to the phosphate of the molecular nucleotide as the rhombus above the first base character 352. However, the empty circle with the gray outline represents a 2-fluororibose synthetic modification to the sugar of the molecular nucleotide.

In some cases, the user interface presentation 300 can present the synthetic modification to the base of the molecular nucleotide using a symbol having a fill pattern that is different from a fill pattern of the symbol representing the synthetic modification to the sugar and a fill pattern of the symbol representing the synthetic modification to the phosphate. For example, as illustrated in FIG. 3, the group of symbols 341 above the second base character 342 (e.g., “u”) includes a first symbol, e.g., a filled circle, and a second symbol, e.g., an empty circle with a black outline. The empty circle with the black outline represents the same synthetic modification to the sugar of the molecular nucleotide as the empty circle with the black outline above the first base character 352. However, the filled circle represents the pseudouracil synthetic modification to the uracil base of the molecular nucleotide. The user interface presentation 300 can generally distinguish the synthetic modifications to bases of molecular nucleotides in any other appropriate manner.

In some implementations, the bioinformatics platform can obtain user-specified visual customizations for the synthetic modifications to the one or more components of the one or more molecular nucleotides in the sequence of molecular nucleotides. The user-specified visual customizations can specify, e.g., different display colors for different types of synthetic modifications to the components of one or more molecular nucleotides in the sequence of molecular nucleotides. The bioinformatics platform can update the user interface presentation according to the user-specified visual customizations.

As a particular example, a user of an end-user device (e.g., the end-user device 150 in FIG. 1) can provide user-specified visual customizations as an input to the bioinformatics platform through the end-user device. Then, instead of representing, e.g., the 2-fluororibose synthetic modification to the sugar of the molecular nucleotide using an empty circle with a gray outline, the bioinformatics platform can update the user interface presentation to represent this synthetic modification using an empty circle having a different color outline in accordance with the user-specified visual customizations. Generally, the user-specified visual customizations can specify any appropriate visual customizations for the synthetic modifications presented in the user interface presentation 300. In some cases, the user interface presentation 300 can further include a legend 310 that presents each symbol and a respective synthetic modification represented by the symbol.

In some implementations, the bioinformatics platform can receive a request to zoom in within the user interface presentation 300. For example, a user of the end-user device can provide the request by interacting with a zoom control 320 in the user interface presentation 300. In response to the request, the bioinformatics platform can transition the user interface presentation 300 to a different display format. The different display format of the user interface presentation 300 is described in more detail next.

FIG. 4 illustrates a different display format 400 of the example user interface presentation (e.g., the user interface presentation 115 in FIG. 1, or the user interface presentation 300 in FIG. 3). In the example of FIG. 4, the different display format 400 presents a sequence of molecular nucleotides (e.g., the same sequence of molecular nucleotides as described above with reference to FIG. 3) that includes 57 molecular nucleotides, each molecular nucleotide having a corresponding position in the sequence indicated by a number, e.g., 1, 2, 3, etc. As described above, the bioinformatics platform can transition the user interface presentation to the different display format 400 in response to a request, e.g., a request provided by a user through the end-user device by interacting with a zoom interface control. FIG. 4 illustrates exploded views 415a and 415b for clarity.

Similarly to the user interface presentation described above with reference to FIG. 3, the different display format can present, for each base in the sequence of molecular nucleotides, a base character that represents the base of the molecular nucleotide. For example, a first base character 430 (“a”) represents a molecular nucleotide (e.g., positioned at the 7^{th} position in the molecular nucleotide sequence) having adenine base. A second base character 440 (“u”) represents a molecular nucleotide (e.g., positioned at the 13^{th} position in the molecular nucleotide sequence) having uracil base. Furthermore, the different display format 400 can similarly include a legend 410 that presents different symbols and respective synthetic modifications represented by the symbols, and a zoom user interface control 422.

The different display format 400 can present the synthetic modification to the sugar of the molecular nucleotide using an outline of the base character that represents the base of the molecular nucleotide. For example, as illustrated in FIG. 4, the first base character 430 (e.g., “a”) includes an outline 432, e.g., a black circle, that represents synthetic modification to the sugar of the molecular nucleotide. Similarly, the second base character 440 (e.g., “u”) includes an outline 442 that is also a black circle and represents the same synthetic modification to the sugar of the molecular nucleotide.

The different display format 400 can present the synthetic modification to the base of the molecular nucleotide using a shaded background of the base character that represents the base of the molecular nucleotide. For example, as illustrated in FIG. 4, the second base character 440 (e.g., “u”) includes a shaded background 445 that represents the pseudouracil synthetic modification to the uracil base of the molecular nucleotide. The different display format 400 can present the synthetic modification to the phosphate of the molecular nucleotide using a single symbol adjacent to the base character that represents the base of the molecular nucleotide. For example, as illustrated in FIG. 4, the first base character 430 (e.g., “a”) includes a rhombus adjacent to the base character (e.g., “a”) that represents the phosphorothioate synthetic modification to the phosphate of the molecular nucleotide. Generally, the different display format 400 can present the synthetic modifications to one or more components of one or more molecular nucleotides in the sequence in any other appropriate manner.

In some implementations, the user interface presentation described above with reference to FIG. 3, and/or the different display format 400 of the user interface presentation, can additionally present user-customizable grouping information for subsequences of the sequence of molecular nucleotides. For example, as illustrated in FIG. 4, the grouping information can indicate a first subsequence 450 of the sequence of molecular nucleotides that includes molecular nucleotides from position 48 to position 57. The grouping information can further indicate a second subsequence 460 of the sequence of molecular nucleotides that includes molecular nucleotides from position 39 to position 47. The user interface presentation can include user interface elements for editing labels for the subsequences and a beginning and an end of the subsequences of the sequence of molecular nucleotides. A user of the user-interface device can interact with the user interface elements to edit labels for the subsequences 450, 460 (e.g., to specify names for each subsequence) and to edit the beginning and the end of each subsequence. For example, the user can specify which molecular nucleotides, and how many molecular nucleotides are included in the subsequence 450, 460.

Example user interface controls for selecting additional, or alternative, synthetic modifications to be presented in the user interface presentation 300, or the new display format 400 of the user interface presentation, are described in more detail next.

FIG. 5 illustrates example user interface controls 500 for selecting additional, or alternative, synthetic modifications to one or more components of one or more molecular nucleotides in a sequence of molecular nucleotides that is presented in a user interface presentation (e.g., the user interface presentation 115 in FIG. 1). In some cases, the user interface controls 500 can be presented in the user interface presentation in addition to the sequence of molecular nucleotides.

As illustrated in FIG. 5, each molecular nucleotide in the sequence of molecular nucleotides can have a respective position in the sequence indicated by a number in the user interface presentation, e.g., 1, 2, 3, etc. The user interface presentation can further specify the sugar, or synthetically modified sugar, of each molecular nucleotide in the sequence. Moreover, the user interface presentation can further specify the phosphate, or synthetically modified phosphate, of each molecular nucleotide in the sequence. A user of the bioinformatics platform is able to search and select a particular synthetic modification from a synthetic modification database (e.g., the synthetic modification database 180 in FIG. 1) for each molecular nucleotide individually.

In response to receiving the selection, the bioinformatics platform can modify the group of symbols adjacent to the base character that represents the base of the molecular nucleotide to represent the one or more additional, or alternative, synthetic modifications in the user interface presentation.

In some implementations, a user of the bioinformatics platform can select one or more molecular nucleotides at one or more respective positions in the sequence, as indicated by numbers in the user interface presentation (e.g., a molecular nucleotide at position 1 and a molecular nucleotide at position 5). Then, the user can select a particular synthetic modification from the synthetic modification database for the selected one or more molecular nucleotides. In response, the bioinformatics platform can simultaneously modify the group of symbols adjacent to the base characters of these one or more molecular nucleotides to represents the selected synthetic modification. In other words, the bioinformatics platform can enable (e.g., simultaneous) bulk editing of a number of molecular nucleotides, e.g., 2, 5, 10, 100, or any other appropriate number of molecular nucleotides, in the user interface presentation.

In some implementations, a user of the bioinformatics platform can use a “find and replace” function in the bioinformatics platform to edit a number of molecular nucleotides in the user interface presentation. For example, the user can use a “search” user interface control (e.g., as illustrated in FIG. 5) to select particular molecular nucleotides that satisfy a predetermined criterion. The predetermined criterion can generally include any appropriate criterion, e.g., molecular nucleotides having a particular base (e.g., the adenine base), molecular nucleotides having one or more components with the same synthetic modification (e.g., the phosphorothioate synthetic modification), or any other appropriate criterion. In response, the bioinformatics platform can (e.g., simultaneously) select all molecular nucleotides in the user interface presentation that satisfy the predetermined criterion. Then, the user can select a particular synthetic modification from the synthetic modification database for the selected molecular nucleotides and the bioinformatics can simultaneously modify the group of symbols adjacent to the base characters of these molecular nucleotides to represents the selected synthetic modification.

In some cases, the bioinformatics platform can enable a user to not only select additional, or alternative, synthetic modifications, but also alternative bases of the molecular nucleotides. For example, the user can use any of the functionalities of the bioinformatics platform described above to modify the base characters in the user interface presentation. As a particular example, the user can select one or more molecular nucleotides in the sequence having, e.g., the adenine base, represented by the base character “a,” and simultaneously modify the one or more molecular nucleotides to have, e.g., the cytosine base, represented by the base character “c,” in the user interface presentation.

An example process for generating the user interface presentation of a sequence of molecular nucleotides is described in more detail next.

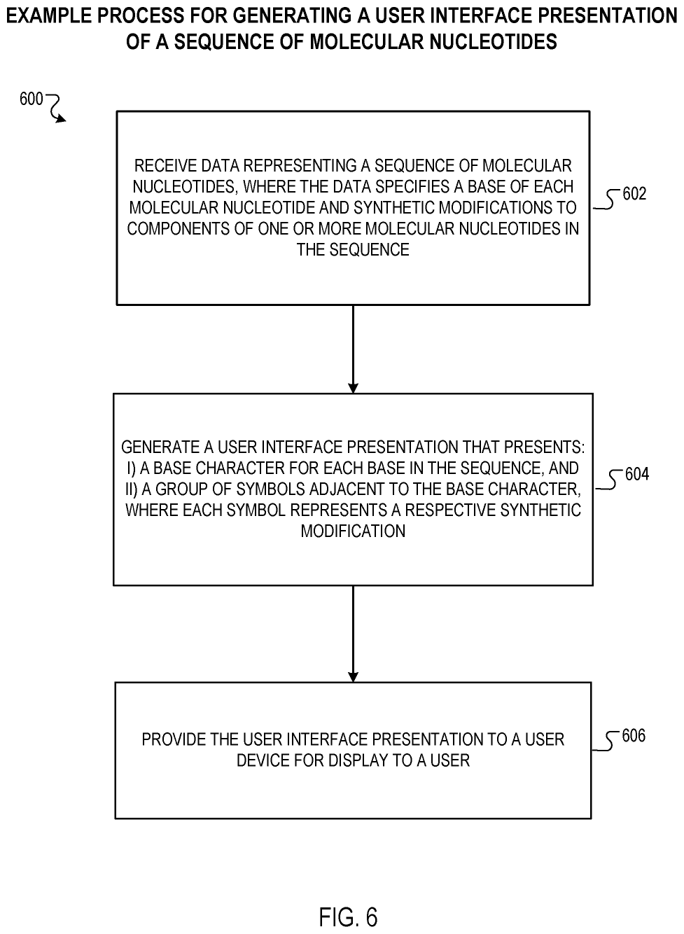

FIG. 6 is a flow diagram of an example process 600 for generating a user interface presentation of a sequence of molecular nucleotides. For convenience, the process 600 is described as being performed by a system of one or more computers located in one or more locations. For example, a bioinformatics platform, e.g., the bioinformatics platform 100 of FIG. 1, appropriately programmed in accordance with this specification, can perform the process 600.

The system receives data representing a sequence of molecular nucleotides (602). The data can specify a base of each molecular nucleotide in the sequence of molecular nucleotides and synthetic modifications to one or more components of one or more molecular nucleotides in the sequence of molecular nucleotides. In some cases, the data representing the sequence of molecular nucleotides can be in a first format. In such cases, the system can receive a user-specified mapping between the first format and a second format, and automatically convert the data into the second format according to the user-specified mapping.

The system generates a user interface presentation (604). The user interface presentation can present: i) for each base in the sequence of molecular nucleotides, a base character that represents the base of the molecular nucleotide, and ii) for each of the one or more molecular nucleotides having one or more components that are synthetically modified, a group of symbols adjacent to the base character that represents the base of the molecular nucleotide, where each symbol in the group of symbols represents a respective synthetic modification. The group of symbols can be presented as a group of vertically stacked symbols and can be positioned above the base character that represents the base of the molecular nucleotide.

In some cases, the group of vertically stacked symbols can include visually distinguishable symbols to represent different synthetic modifications. For example, each symbol included in the group of vertically stacked symbols can have a respective shape to represent the respective synthetic modification that is different from the shapes of the other symbols in the group of vertically stacked symbols. As a particular example, the components that are synthetically modified can include sugar and phosphate. In such cases, the synthetic modification to the sugar can be represented using a symbol having a first shape and the synthetic modification to the phosphate can be represented using a symbol having a second different shape. As another particular example, the components that are synthetically modified can further include the base of the molecular nucleotide. In such cases, the synthetic modification to the base can be represented using a symbol having a fill pattern that is different from a fill pattern of the symbol representing the synthetic modification to the sugar and a fill pattern of the symbol representing the synthetic modification to the phosphate.

In some cases, the system can present, in the user interface presentation, user-customizable grouping information for subsequences of the sequence of molecular nucleotides.

In some cases, the system can generate the user interface presentation that includes user interface elements for editing labels for the subsequences and a beginning and an end of the subsequences of the sequence of molecular nucleotides.

The system provides the user interface presentation to a user device for display to a user (606).

In some implementations, the system can receive a request to zoom in within the user interface presentation and, in response, transition the user interface presentation to a different display format. The different display format can present a modified user interface presentation. For example, if the components that are synthetically modified include a sugar, the synthetic modification to the sugar can be represented in the different display format using an outline of the base character that represents the base of the molecular nucleotide. As another example, if the one or more components that are synthetically modified further include the base of the molecular nucleotide, the synthetic modification to the base can be represented in the different display format using a shaded background of the base character that represents the base of the molecular nucleotide. As yet another example, if the components that are synthetically modified further include a phosphate, the synthetic modification to the phosphate can be represented in the different display format using a single symbol adjacent to the base character that represents the base of the molecular nucleotide.

In some implementations, the system can update the user interface presentation using user-specified visual customizations of the synthetic modifications. For example, the system can obtain user-specified visual customizations for the synthetic modifications to the one or more components of the one or more molecular nucleotides, and update the user interface presentation, or the different display format, according to the user-specified visual customizations. The user-specified visual customizations can specify different display colors for different types of synthetic modifications to the one or more components of the one or more molecular nucleotides in the sequence of molecular nucleotides.

In some implementations, the system can compare sequences of molecular nucleotides and modify the user interface presentation based on the comparison. For example, the system can receive a request to compare a sequence of molecular nucleotides to a second sequence of molecular nucleotides. Then, the system can determine that a first nucleotide in the sequence of molecular nucleotides has a synthetic modification that is not present in a second nucleotide at a corresponding position in the second sequence of molecular nucleotides. Based on the comparison, the system can represent, in the user interface presentation, the synthetic modification that is not present in the second nucleotide using a symbol that is visually distinguished from other symbols in the user interface presentation.

In some implementations, the system can present a user interface control for selecting one or more additional synthetic modifications. The system can receive a selection to present the one or more additional synthetic modifications, e.g., as user input into the end-user device. In response to receiving the selection, the system can modify the group of symbols adjacent to the base character that represents the base of the molecular nucleotide to represent the one or more additional synthetic modifications.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method comprising:
receiving data representing a sequence of molecular nucleotides, wherein the data specifies a base of each molecular nucleotide in the sequence of molecular nucleotides and synthetic modifications to one or more components of one or more molecular nucleotides in the sequence of molecular nucleotides;
generating a user interface presentation that presents:
i) for each base in the sequence of molecular nucleotides, a base character that represents the base of the molecular nucleotide, and
ii) for each of the one or more molecular nucleotides having one or more components that are synthetically modified, a group of symbols adjacent to the base character that represents the base of the molecular nucleotide, wherein each symbol in the group of symbols represents a respective synthetic modification; and
providing the user interface presentation to a user device for display to a user.

2. The method of claim 1, wherein the group of symbols is presented as a group of vertically stacked symbols.

3. The method of claim 2, wherein the group of symbols is presented above the base character that represents the base of the molecular nucleotide.

4. The method of claim 2, wherein the group of vertically stacked symbols comprises visually distinguishable symbols to represent different synthetic modifications.

5. The method of claim 4, wherein each symbol included in the group of vertically stacked symbols has a respective shape to represent the respective synthetic modification that is different from the shapes of the other symbols in the group of vertically stacked symbols.

6. The method of claim 5, wherein the one or more components that are synthetically modified comprise a sugar and a phosphate, and wherein the synthetic modification to the sugar is represented using a symbol having a first shape and the synthetic modification to the phosphate is represented using a symbol having a second different shape.

7. The method of claim 6, wherein the one or more components that are synthetically modified further comprise the base of the molecular nucleotide, and wherein the synthetic modification to the base is represented using a symbol having a fill pattern that is different from a fill pattern of the symbol representing the synthetic modification to the sugar and a fill pattern of the symbol representing the synthetic modification to the phosphate.

8. The method of claim 1, further comprising:
receiving a request to zoom in within the user interface presentation; and
in response, transitioning the user interface presentation to a different display format.

9. The method of claim 8, wherein the one or more components that are synthetically modified comprise a sugar, and wherein the synthetic modification to the sugar is represented in the different display format using an outline of the base character that represents the base of the molecular nucleotide.

10. The method of claim 9, wherein the one or more components that are synthetically modified further comprise the base of the molecular nucleotide, and wherein the synthetic modification to the base is represented in the different display format using a shaded background of the base character that represents the base of the molecular nucleotide.

11. The method of claim 10, wherein the one or more components that are synthetically modified further comprise a phosphate, and wherein the synthetic modification to the phosphate is represented in the different display format using a single symbol adjacent to the base character that represents the base of the molecular nucleotide.

12. The method of claim 8, further comprising:
obtaining user-specified visual customizations for the synthetic modifications to the one or more components of the one or more molecular nucleotides in the sequence of molecular nucleotides; and
updating the user interface presentation or the different display format of the user interface presentation according to the user-specified visual customizations.

13. The method of claim 12, wherein the user-specified visual customizations specify different display colors for different types of synthetic modifications to the one or more components of the one or more molecular nucleotides in the sequence of molecular nucleotides.

14. The method of claim 1, wherein the data representing the sequence of molecular nucleotides is in a first format, and wherein the method further comprises:
receiving a user-specified mapping between the first format and a second format; and
automatically converting the data into the second format according to the user-specified mapping.

15. The method of claim 1, further comprising:
receiving a request to compare the sequence of molecular nucleotides to a second sequence of molecular nucleotides;
determining that a first nucleotide in the sequence of molecular nucleotides has a synthetic modification that is not present in a second nucleotide at a corresponding position in the second sequence of molecular nucleotides; and
representing in the user interface presentation the synthetic modification that is not present in the second nucleotide using a symbol that is visually distinguished from other symbols in the user interface presentation.

16. The method of claim 1, wherein the user interface presentation presents user-customizable grouping information for subsequences of the sequence of molecular nucleotides.

17. The method of claim 16, wherein the user interface presentation includes user interface elements for editing labels for the subsequences and a beginning and an end of the subsequences of the sequence of molecular nucleotides.

18. The method of claim 1, further comprising:
presenting a user interface control for selecting one or more additional synthetic modifications;
receiving a selection to present the one or more additional, or alternative, synthetic modifications; and
in response to receiving the selection, modifying the group of symbols adjacent to the base character that represents the base of the molecular nucleotide to represent the one or more additional, or alternative, synthetic modifications.

19. A system comprising:
one or more computers; and
one or more storage devices communicatively coupled to the one or more computers, wherein the one or more storage devices store instructions that, when executed by the one or more computers, cause the one or more computers to perform operations comprising:
receiving data representing a sequence of molecular nucleotides, wherein the data specifies a base of each molecular nucleotide in the sequence of molecular nucleotides and synthetic modifications to one or more components of one or more molecular nucleotides in the sequence of molecular nucleotides;
generating a user interface presentation that presents:
i) for each base in the sequence of molecular nucleotides, a base character that represents the base of the molecular nucleotide, and
ii) for each of the one or more molecular nucleotides having one or more components that are synthetically modified, a group of symbols adjacent to the base character that represents the base of the molecular nucleotide, wherein each symbol in the group of symbols represents a respective synthetic modification; and
providing the user interface presentation to a user device for display to a user.

20. One or more non-transitory computer storage media storing instructions that when executed by one or more computers cause the one or more computers to perform operations comprising:
receiving data representing a sequence of molecular nucleotides, wherein the data specifies a base of each molecular nucleotide in the sequence of molecular nucleotides and synthetic modifications to one or more components of one or more molecular nucleotides in the sequence of molecular nucleotides;
generating a user interface presentation that presents:
i) for each base in the sequence of molecular nucleotides, a base character that represents the base of the molecular nucleotide, and
ii) for each of the one or more molecular nucleotides having one or more components that are synthetically modified, a group of symbols adjacent to the base character that represents the base of the molecular nucleotide, wherein each symbol in the group of symbols represents a respective synthetic modification; and
providing the user interface presentation to a user device for display to a user.
